(19) United States
(12) Patent Application Publication
(10) Pub. No.: US 2023/0383351 A1
(43) Pub. Date: Nov. 30, 2023

(54) Rare Variants In Hematopoietic Stem Cells (HSC) And Hematopoietic Progenitor Cells (HPC) Associated With Somatic Alterations Of The Blood

(71) Applicant: Regeneron Pharmaceuticals, Inc., Tarrytown, NY (US)

(72) Inventors: Michael D. Kessler, Tarrytown, NY (US); Hyun Min Kang, Tarrytown, NY (US); Eric Jorgenson, Tarrytown, NY (US)

(21) Appl. No.: 18/303,229

(22) Filed: Apr. 19, 2023

Related U.S. Application Data
(60) Provisional application No. 63/333,697, filed on Apr. 22, 2022.

(51) Int. Cl. C12Q 1/6883; C12Q 1/6869

(52) U.S. Cl. CPC C12Q 1/6883; C12Q 1/6869; C12Q 2600/158; C12Q 2600/156

(57) ABSTRACT

Methods of treating, preventing, or reducing somatic alterations of the blood of a subject with kinetochore associated 1 (KNTC1) antagonists, ring finger and CCCH-type domains 1 (RC3H1) agonists, YLP motif containing 1 (YLPM1) agonists, Major Histocompatibility Complex, Class II, and/or DR beta 5 (HLA-DRB5) agonists are provided herein. Methods of treating a subject with a therapeutic agent that treats, prevents, or reduces somatic alterations of the blood are also provided. Methods of identifying a subject having an increased risk of developing somatic alterations of the blood are also provided.

### FIELD

The present disclosure relates generally to the treatment, prevention, or reduction of somatic alterations of the blood of a subject with kinetochore associated 1(KNTC1) antagonists, ring finger and CCCH-type domains 1 (RC3H1) agonists, YLP motif containing 1 (YLPM1) agonists, Major Histocompatibility Complex, Class II, and/or DR beta 5 (HLA-DRB5) agonists, the treatment of a subject with a therapeutic agent that treats, prevents, or reduces somatic alterations of the blood, and the identification of a subject having an increased risk of developing somatic alterations of the blood.

### BACKGROUND

As humans age, somatic alterations accrue in the DNA of hematopoietic stem cells (HSC) or hematopoietic progenitor cells (HPC) leading to mitotic errors and DNA damage. Alterations that confer a selective growth advantage can lead to expansion of selective cell lineages. Somatic alterations in the blood are associated with clonal hematopoiesis (CH) or leukocyte telomere length (LTL) reductions.

The presence of CH has been associated with an increased risk of hematological neoplasms, cytopenias, cardiovascular disease, infection, and all-cause mortality (Jaiswal et al., N. Engl. J. Med., 2014, 371, 2488-98; Jaiswal et al., N. Engl. J. Med., 2017, 377, 111-21; Jaiswal & Ebert, Science, 2019, 366, eaan467; Zekavat et al., Nat. Med., 2021, 27, 1012-24; Niroula et al., Nat. Med., 2021, 27, 1921-27).

A mosaic loss of chromosome Y (mLOY) in blood cells is one of the most frequent chromosome alterations in adult males. mLOY is strongly associated with CH, hematopoietic malignancies, other hematopoietic diseases, and other nonhematopoietic diseases (Zhang et al., JCI Insight, 2022, 7, e153768).

Mosaic chromosomal alterations (mCAs), which are detected from genotyping of blood-derived DNA, are structural somatic variants indicative of CH and are associated with aberrant leukocyte cell counts, hematological malignancy, and mortality (Zekavat et al., Nat. Med., 2021, 27, 1012-24).

Telomeres, the end fragments of chromosomes, play key roles in cellular proliferation and senescence. The genetic architecture of naturally-occurring variations in leukocyte telomere length (LTL) have been characterized and causal links between LTL and biomedical phenotypes have been identified (Codd et al., Nat. Genet., 2021, 53, 1425-33).

KNTC1 plays a role in mitotic checkpoint activity (Scaerou et al., J. Cell Sci., 2001, 114, 3103-14). Targeted knockdown of KNTC1 has been shown to antagonize cell proliferation and induce apoptosis across numerous cancer cell types (Zhengxiang et al., 2021, 3 Biotech, 11, 1-11; Huang et al., Crit. Rev. Eukaryot. Gene Expr., 2021, 31, 49-60; Liu et al., Int. J. Oncol., 2019, 54, 1053-60).

RC3H1 is a regulator of inflammation and immune homeostasis (Schaefer & Klein, Genes Immun., 2016, 17, 79-84). RC3H1 is directly associated with angioimmunoblastic T-cell lymphoma in mice (Chiba & Yanagimoto, Leukemia, 2020, 34, 2592-606).

YLPM1 has been shown to limit telomerase activity by downregulating TERT by promoter binding and YLPM1 function loss by point mutation and/or mosaic chromosomal alteration likely drives clonal hematopoiesis.

HLA-DRB5 is approximately 26-28 kDa and belongs to the class of HLA class II beta chain paralogues. Within the DR molecule, the beta chain contains all the polymorphisms specifying the peptide binding specificities (see, world wide web at “pharos.nih.gov/targets/Q30154”).

### SUMMARY

The present disclosure provides methods of treating, preventing, or reducing the development of CH in a subject, the methods comprising administering at least one KNTC1 antagonist to the subject.

The present disclosure also provides methods of treating, preventing, or reducing the development of CH in a subject, the methods comprising administering at least one RC3H1 agonist to the subject.

The present disclosure also provides methods of treating, preventing, or reducing the development of CH in a subject, the methods comprising administering at least one YLPM1 agonist to the subject.

The present disclosure also provides methods of treating, preventing, or reducing the development of LTL reduction in a subject, the methods comprising administering at least one HLA-DRB5 agonist.

The present disclosure also provides methods of treating a subject with a therapeutic agent that treats, prevents, or reduces development of CH, wherein the subject has CH or is at risk of developing CH, the methods comprising: determining whether the subject has a KNTC1 variant nucleic acid molecule by: obtaining or having obtained a biological sample from the subject; and performing or having performed a sequence analysis on the biological sample to determine if the subject has a genotype comprising a KNTC1 variant nucleic acid molecule; and administering or continuing to administer the therapeutic agent that treats, prevents, or reduces development of CH in an amount that is the same as or greater than a standard dosage amount to a subject that is KNTC1 reference; and/or administering a KNTC1 antagonist to the subject; administering or continuing to administer the therapeutic agent that treats, prevents, or reduces development of CH in an amount that is the same as or less than a standard dosage amount to a subject that is heterozygous for the KNTC1 variant nucleic acid molecule; and/or administering a KNTC1 antagonist to the subject; or administering or continuing to administer the therapeutic agent that treats, prevents, or reduces development of CH in an amount that is the same as or less than a standard dosage amount to a subject that is homozygous for the KNTC1 variant nucleic acid molecule; and/or administering a KNTC1 antagonist to the subject; wherein the presence of a genotype having the KNTC1 variant nucleic acid molecule indicates the subject has a decreased risk of developing CH.

The present disclosure also provides methods of treating a subject with a therapeutic agent that treats, prevents, or reduces development of CH, wherein the subject has CH or is at risk of developing CH, the methods comprising: determining whether the subject has an RC3H1 variant nucleic acid molecule by: obtaining or having obtained a biological sample from the subject; and performing or having performed a sequence analysis on the biological sample to determine if the subject has a genotype comprising an RC3H1 variant nucleic acid molecule; and administering or continuing to administer the therapeutic agent that treats, prevents, or reduces development of CH in an amount that is the same as or less than a standard dosage amount to a subject that is RC3H1 reference; and/or administering an RC3H1 agonist to the subject; administering or continuing to administer the therapeutic agent that treats, prevents, or reduces development of CH in an amount that is the same as or greater than a standard dosage amount to a subject that is heterozygous for the RC3H1 variant nucleic acid molecule; and/or administering an RC3H1 agonist to the subject; or administering or continuing to administer the therapeutic agent that treats, prevents, or reduces development of CH in an amount that is the same as or greater than a standard dosage amount to a subject that is homozygous for the RC3H1 variant nucleic acid molecule; and/or administering an RC3H1 agonist to the subject; wherein the presence of a genotype having the RC3H1 variant nucleic acid molecule indicates the subject has an increased risk of developing CH.

The present disclosure also provides methods of treating a subject with a therapeutic agent that treats, prevents, or reduces development of CH, wherein the subject has CH or is at risk of developing CH, the methods comprising: determining whether the subject has a YLPM1 variant nucleic acid molecule by: obtaining or having obtained a biological sample from the subject; and performing or having performed a sequence analysis on the biological sample to determine if the subject has a genotype comprising a YLPM1 variant nucleic acid molecule; and administering or continuing to administer the therapeutic agent that treats, prevents, or reduces development of CH in an amount that is the same as or less than a standard dosage amount to a subject that is YLPM1 reference; and/or administering a YLPM1 agonist to the subject; administering or continuing to administer the therapeutic agent that treats, prevents, or reduces development of CH in an amount that is the same as or greater than a standard dosage amount to a subject that is heterozygous for the YLPM1 variant nucleic acid molecule; and/or administering a YLPM1 agonist to the subject; or administering or continuing to administer the therapeutic agent that treats, prevents, or reduces development of CH in an amount that is the same as or greater than a standard dosage amount to a subject that is homozygous for the YLPM1 variant nucleic acid molecule; and/or administering a YLPM1 agonist to the subject; wherein the presence of a genotype having the YLPM1 variant nucleic acid molecule indicates the subject has an increased risk of developing CH.

The present disclosure also provides methods of treating a subject with a therapeutic agent that treats, prevents, or reduces development of LTL reduction, wherein the subject has LTL reduction or is at risk of developing LTL reduction, the methods comprising: determining whether the subject has an HLA-DRB5 variant nucleic acid molecule by: obtaining or having obtained a biological sample from the subject; and performing or having performed a sequence analysis on the biological sample to determine if the subject has a genotype comprising an HLA-DRB5 variant nucleic acid molecule; and administering or continuing to administer the therapeutic agent that treats, prevents, or reduces development of LTL reduction in an amount that is the same as or less than a standard dosage amount to a subject that is HLA-DRB5 reference; and/or administering an HLA-DRB5 agonist to the subject; administering or continuing to administer the therapeutic agent that treats, prevents, or reduces development of LTL reduction in an amount that is the same as or greater than a standard dosage amount to a subject that is heterozygous for the HLA-DRB5 variant nucleic acid molecule; and/or administering an HLA-DRB5 agonist to the subject; or administering or continuing to administer the therapeutic agent that treats, prevents, or reduces development of LTL reduction in an amount that is the same as or greater than a standard dosage amount to a subject that is homozygous for the HLA-DRB5 variant nucleic acid molecule; and/or administering an HLA-DRB5 agonist to the subject; wherein the presence of a genotype having the HLA-DRB5 variant nucleic acid molecule indicates the subject has an increased risk of developing LTL.

The present disclosure also provides methods of identifying a subject having an increased risk of developing CH, the methods comprising: determining or having determined the presence or absence of a KNTC1 variant nucleic acid molecule in a biological sample obtained from the subject; wherein: when the subject is KNTC1 reference, then the subject has an increased risk of developing CH compared to a subject that comprises the KNTC1 variant nucleic acid molecule; and when the subject is heterozygous or homozygous for the KNTC1 variant nucleic acid molecule, then the subject has an decreased risk of developing CH compared to a subject that is KNTC1 reference.

The present disclosure also provides methods of identifying a subject having an increased risk of developing CH, the methods comprising: determining or having determined the presence or absence of an RC3H1 variant nucleic acid molecule in a biological sample obtained from the subject; wherein: when the subject is RC3H1 reference, then the subject has a decreased risk of developing CH compared to a subject that comprises the RC3H1 variant nucleic acid molecule; and when the subject is heterozygous or homozygous for the RC3H1 variant nucleic acid molecule, then the subject has an increased risk of developing CH compared to a subject that is RC3H1 reference.

The present disclosure also provides methods of identifying a subject having an increased risk of developing CH, the methods comprising: determining or having determined the presence or absence of a YLPM1 variant nucleic acid molecule in a biological sample obtained from the subject; wherein: when the subject is YLPM1 reference, then the subject has a decreased risk of developing CH compared to a subject that comprises the YLPM1 variant nucleic acid molecule; and when the subject is heterozygous or homozygous for the YLPM1 variant nucleic acid molecule, then the subject has an increased risk of developing CH compared to a subject that is YLPM1 reference.

The present disclosure also provides methods of identifying a subject having an increased risk of developing LTL reduction, the methods comprising: determining or having determined the presence or absence of an HLA-DRB5 variant nucleic acid molecule in a biological sample obtained from the subject; wherein: when the subject is HLA-DRB5 reference, then the subject has a decreased risk of developing LTL reduction compared to a subject that comprises the HLA-DRB5 variant nucleic acid molecule; and when the subject is heterozygous or homozygous for the HLA-DRB5 variant nucleic acid molecule, then the subject has an increased risk of developing LTL reduction compared to a subject that is HLA-DRB5 reference.

### BRIEF DESCRIPTION OF THE DRAWINGS

The accompanying FIGURES, which are incorporated in and constitute a part of this specification, illustrate several features of the present disclosure.

The patent or application file contains at least one drawing executed in color. Copies of this patent or patent application publication with color drawing(s) will be provided by the Office upon request and payment of the necessary fee.

FIG. 1 shows KNTC1 expression across tissue types. Barplots of expression level (transcript per million, abscissa) for the KNTC1 gene across tissues, as ascertained by Genotype-Tissue Expression (GTEx) project via bulk RNA Sequencing are shown.

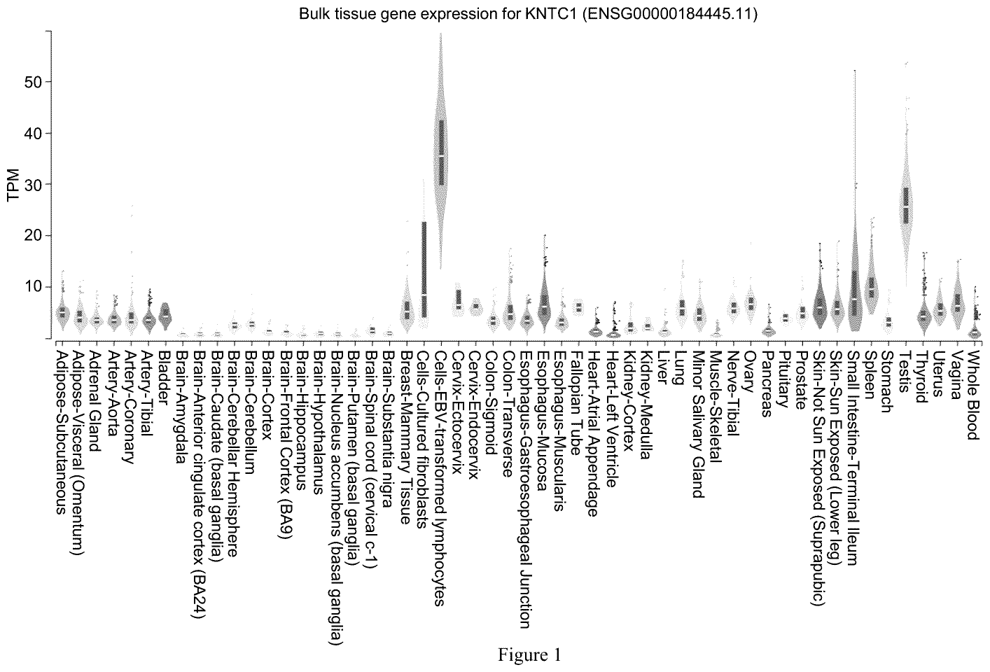

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or Alternately phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human. In some embodiments, the human is a patient under the care of a physician.

It has been observed in accordance with the present disclosure that a rare missense variant in the KNTC1 gene indicates that a subject having mLOY has a decreased risk of developing CH. It has also been observed in accordance with the present disclosure that a rare loss-of-function variant in the RC3H1 gene indicates that a subject having mCA has an increased risk of developing CH. It has also been observed in accordance with the present disclosure that a rare loss-of-function variant in the YLPM1 gene indicates that a subject having mCA has an increased risk of developing CH. It has also been observed in accordance with the present disclosure that a subject having a rare loss-of-function variant in the HLA-DRB5 gene has an increased risk of developing LTL reduction.

The present disclosure provides methods of treating, preventing, or reducing the development of a somatic alteration of the blood of a subject, the methods comprising administering at least one KNTC1 antagonist, at least one RC3H1 agonist, at least one YLPM1 agonist, and/or at least one HLA-DRB5 agonist to the subject. In some embodiments, the somatic alteration is associated with CH or LTL reduction. In some embodiments, the subject comprises an mLOY and/or an mCA. In some embodiments, the subject comprises an mLOY. In some embodiments, the subject comprises an mCA. In some embodiments, the somatic alteration comprises an LTL reduction. In some embodiments, the methods comprise administering at least one KNTC1 antagonist. In some embodiments, the methods comprise administering at least one RC3H1 agonist. In some embodiments, the methods comprise administering at least one YLPM1 agonist. In some embodiments, the methods comprise administering at least one HLA-DRB5 agonist. In some embodiments, when the somatic alteration is associated with CH, the subject is administered at least one KNTC1 antagonist, at least one RC3H1 agonist, and/or at least one YLPM1 agonist. In some embodiments, when the subject comprises mLOY, the subject is administered at least one KNTC1 antagonist. In some embodiments, when the subject comprises mCA, the subject is administered at least one RC3H1 agonist and/or at least one YLPM1 agonist. In some embodiments, when the subject comprises mCA, the subject is administered at least one RC3H1 agonist. In some embodiments, when the subject comprises mCA, the subject is administered at least one YLPM1 agonist. In some embodiments, when the somatic alteration is associated with LTL reduction, the subject is administered at least one HLA-DRB5 agonist.

The present disclosure also provides methods of treating, preventing, or reducing the development of CH in a subject, the method comprising administering at least one KNTC1 antagonist to the subject. In some embodiments, the subject comprises mLOY and/or mCA. In some embodiments, the subject comprises mLOY. In some embodiments, the subject comprises mCA.

The present disclosure also provides methods of treating, preventing, or reducing the development of CH in a subject, the method comprising administering at least RC3H1 agonist to the subject. In some embodiments, the subject comprises mLOY and/or mCA. In some embodiments, the subject comprises mLOY. In some embodiments, the subject comprises mCA.

The present disclosure also provides methods of treating, preventing, or reducing the development of CH in a subject, the method comprising administering at least one YLPM1 agonist to the subject. In some embodiments, the subject comprises mLOY and/or mCA. In some embodiments, the subject comprises mLOY. In some embodiments, the subject comprises mCA.

The present disclosure also provides methods of treating, preventing, or reducing the development of LTL reduction in a subject, the method comprising administering at least one HLA-DRB5 agonist.

The present disclosure also provides methods of treating a subject with a therapeutic agent that treats, prevents, or reduces development of CH. In some embodiments, the subject has CH or is at risk of developing CH. In some embodiments, the methods comprise determining whether the subject has a KNTC1 variant nucleic acid molecule. In some embodiments, the determining comprises obtaining or having obtained a biological sample from the subject. In some embodiments, the determining comprises performing or having performed a sequence analysis on the biological sample to determine if the subject has a genotype comprising a KNTC1 variant nucleic acid molecule. In some embodiments, the methods comprise administering or continuing to administer the therapeutic agent that treats, prevents, or reduces development of CH in an amount that is the same as or greater than a standard dosage amount to a subject that is KNTC1 reference; and/or administering a KNTC1 antagonist to the subject. In some embodiments, the subject comprises mLOY and/or mCA. In some embodiments, the methods comprise administering or continuing to administer the therapeutic agent that treats, prevents, or reduces development of CH in an amount that is the same as or less than a standard dosage amount to a subject that is heterozygous for the KNTC1 variant nucleic acid molecule; and/or administering a KNTC1 antagonist to the subject. In some embodiments, the methods comprise administering or continuing to administer the therapeutic agent that treats, prevents, or reduces development of CH in an amount that is the same as or less than a standard dosage amount to a subject that is homozygous for the KNTC1 variant nucleic acid molecule; and/or administering a KNTC1 antagonist to the subject. In some embodiments, the subject comprises mLOY. In some embodiments, the subject comprises mCA. The presence of a genotype having the KNTC1 variant nucleic acid molecule indicates the subject has a decreased risk of developing CH.

The present disclosure also provides methods of treating a subject with a therapeutic agent that treats, prevents, or reduces development of CH. In some embodiments, the subject has CH or is at risk of developing CH. In some embodiments, the methods comprise determining whether the subject has an RC3H1 variant nucleic acid molecule. In some embodiments, the determining comprises obtaining or having obtained a biological sample from the subject. In some embodiments, the determining comprises performing or having performed a sequence analysis on the biological sample to determine if the subject has a genotype comprising an RC3H1 variant nucleic acid molecule. In some embodiments, the methods comprise administering or continuing to administer the therapeutic agent that treats, prevents, or reduces development of CH in an amount that is the same as or less than a standard dosage amount to a subject that is RC3H1 reference; and/or administering an RC3H1 agonist to the subject. In some embodiments, the methods comprise administering or continuing to administer the therapeutic agent that treats, prevents, or reduces development of CH in an amount that is the same as or greater than a standard dosage amount to a subject that is heterozygous for the RC3H1 variant nucleic acid molecule; and/or administering an RC3H1 agonist to the subject. In some embodiments, the methods comprise administering or continuing to administer the therapeutic agent that treats, prevents, or reduces development of CH in an amount that is the same as or greater than a standard dosage amount to a subject that is homozygous for the RC3H1 variant nucleic acid molecule; and/or administering an RC3H1 agonist to the subject. In some embodiments, the subject comprises mLOY. In some embodiments, the subject comprises mCA. The presence of a genotype having the RC3H1 variant nucleic acid molecule indicates the subject has an increased risk of developing CH. In some embodiments, the CH comprises an mLOY and/or an mCA. In some embodiments, the CH comprises an mLOY. In some embodiments, the CH comprises an mCA.

The present disclosure also provides methods of treating a subject with a therapeutic agent that treats, prevents, or reduces development of CH. In some embodiments, the subject has CH or is at risk of developing CH. In some embodiments, the methods comprise determining whether the subject has a YLPM1 variant nucleic acid molecule. In some embodiments, the determining comprises obtaining or having obtained a biological sample from the subject. In some embodiments, the determining comprises performing or having performed a sequence analysis on the biological sample to determine if the subject has a genotype comprising a YLPM1 variant nucleic acid molecule. In some embodiments, the methods comprise administering or continuing to administer the therapeutic agent that treats, prevents, or reduces development of CH in an amount that is the same as or less than a standard dosage amount to a subject that is YLPM1 reference; and/or administering a YLPM1 agonist to the subject. In some embodiments, the methods comprise administering or continuing to administer the therapeutic agent that treats, prevents, or reduces development of CH in an amount that is the same as or greater than a standard dosage amount to a subject that is heterozygous for the YLPM1 variant nucleic acid molecule; and/or administering a YLPM1 agonist to the subject. In some embodiments, the methods comprise administering or continuing to administer the therapeutic agent that treats, prevents, or reduces development of CH in an amount that is the same as or greater than a standard dosage amount to a subject that is homozygous for the YLPM1 variant nucleic acid molecule; and/or administering a YLPM1 agonist to the subject. In some embodiments, the subject comprises mLOY. In some embodiments, the subject comprises mCA. The presence of a genotype having the YLPM1 variant nucleic acid molecule indicates the subject has an increased risk of developing CH.

The present disclosure also provides methods of treating a subject with a therapeutic agent that treats, prevents, or reduces development of LTL reduction. In some embodiments, the subject has LTL reduction or is at risk of developing LTL reduction. In some embodiments, the methods comprise determining whether the subject comprises an HLA-DRB5 variant nucleic acid molecule. In some embodiments, the determining comprises obtaining or having obtained a biological sample from the subject. In some embodiments, the determining comprises performing or having performed a sequence analysis on the biological sample to determine if the subject has a genotype comprising an HLA-DRB5 variant nucleic acid molecule. In some embodiments, the methods comprise administering or continuing to administer the therapeutic agent that treats, prevents, or reduces development of LTL reduction in an amount that is the same as or less than a standard dosage amount to a subject that is HLA-DRB5 reference; and/or administering an HLA-DRB5 agonist to the subject. In some embodiments, the methods comprise administering or continuing to administer the therapeutic agent that treats, prevents, or reduces development of LTL reduction in an amount that is the same as or greater than a standard dosage amount to a subject that is heterozygous for the HLA-DRB5 variant nucleic acid molecule; and/or administering an HLA-DRB5 agonist to the subject. In some embodiments, the methods comprise administering or continuing to administer the therapeutic agent that treats, prevents, or reduces development of LTL reduction in an amount that is the same as or greater than a standard dosage amount to a subject that is homozygous for the HLA-DRB5 variant nucleic acid molecule; and/or administering an HLA-DRB5 agonist to the subject. In some embodiments, the presence of a genotype having the HLA-DRB5 variant nucleic acid molecule indicates the subject has an increased risk of developing LTL reduction.

In any of the embodiments described herein wherein a subject has CH or is at risk of developing CH, the subject has or is at risk of developing a hematologic cancer, a myeloid neoplasia, a lymphoid neoplasia, an atherosclerotic cardiovascular disease, a coronary heart disease, a myocardial infarction, or a severe calcified aortic valve stenosis. In any of the embodiments described herein, the CH disorder or the CH-related disorder, is a hematologic cancer, a myeloid neoplasia, a lymphoid neoplasia, an atherosclerotic cardiovascular disease, a coronary heart disease, a myocardial infarction, and/or a severe calcified aortic valve stenosis. In any of the embodiments described herein, the CH disorder or the CH-related disorder is a hematologic cancer, a myeloid neoplasia, or a lymphoid neoplasia. In some embodiments, the CH disorder or the CH-related disorder is a hematologic cancer. In some embodiments, the CH disorder is a hematologic cancer. In some embodiments, the CH-related disorder is a hematologic cancer. In some embodiments, the CH disorder or the CH-related disorder is a myeloid neoplasia. In some embodiments, the CH disorder is a myeloid neoplasia. In some embodiments, the CH-related disorder is a myeloid neoplasia. In some embodiments, the CH disorder or the CH-related disorder is a lymphoid neoplasia. In some embodiments, the CH disorder is a lymphoid neoplasia. In some embodiments, the CH-related disorder is a lymphoid neoplasia. In some embodiments, the CH disorder or the CH-related disorder is an atherosclerotic cardiovascular disease. In some embodiments, the CH disorder is an atherosclerotic cardiovascular disease. In some embodiments, the CH-related disorder is an atherosclerotic cardiovascular disease. In some embodiments, the CH disorder or the CH-related disorder is a coronary heart disease. In some embodiments, the CH disorder is a coronary heart disease. In some embodiments, the CH-related disorder is a coronary heart disease. In some embodiments, the CH disorder or the CH-related disorder is a myocardial infarction. In some embodiments, the CH disorder is a myocardial infarction. In some embodiments, the CH-related disorder is a myocardial infarction. In some embodiments, the CH disorder or the CH-related disorder is a severe calcified aortic valve stenosis. In some embodiments, the CH disorder is a severe calcified aortic valve stenosis. In some embodiments, the CH-related disorder is a severe calcified aortic valve stenosis.

In any of the embodiments described herein wherein a subject has LTL reduction or is at risk of developing LTL reduction, the subject has or is at risk of decreased longevity and/or an increased risk of developing one or more age-related diseases. As used herein, the term “longevity” refers to the lifespan of an organism. For example, a human having a lifespan of 100 years is considered to have increased longevity compared to a human having a lifespan of less than 100 years (e.g., 90 years, 80 years, 70 years, or 60 years, or less). Although lifespan can be measured in an individual organism, it is common to measure and compare mean or median lifespan of populations of individual organisms. In addition, the lifespan of a group of individuals that comprise LTL reduction is expected to be shorter than the lifespan of a group of individuals that do not comprise LTL reduction. Any inhibition or delay of development or severity of any of the age-related diseases and/or any increase in longevity is considered to be an inducement of healthy aging. In any of the embodiments described herein, the age-related diseases comprise cardiovascular disease, diabetes, atherosclerosis, obesity, cancer, infection, immunosenescence, coronary artery disease, and neurological disorders (e.g., stable miled cognitive impairment, and major depression disorder). In any of the embodiments described herein, the age-related disease is cardiovascular disease. In any of the embodiments described herein, the age-related disease is diabetes. In any of the embodiments described herein, the age-related disease is atherosclerosis. In any of the embodiments described herein, the age-related disease is immunosenescence. In any of the embodiments described herein, the age-related disease is obesity. In any of the embodiments described herein, the age-related disease is cancer. In any of the embodiments described herein, the age-related disease is infection. In any of the embodiments described herein, the age-related disease is coronary artery disease. In any of the embodiments described herein, the age-related disease is a neurological disorder, such as Alzheimer's disease.

In some embodiments, the subject is administered an RC3H1 agonist, a YLPM1 agonist, and/or an HLA-DRB5 agonist. In some embodiments, the subject is administered an RC3H1 agonist. In some embodiments, the RC3H1 agonist is RC3H1 protein. In some embodiments, the subject is administered a YLPM1 agonist. In some embodiments the YLPM1 agonist is YLPM1 protein. In some embodiments, the subject is administered an HLA-DRB5 agonist. In some embodiments, the HLA-DRB5 agonist is HLA-DRB5 protein.

In some embodiments, the subject is administered a KNTC1 antagonist. In some embodiments, the KNTC1 antagonist comprises an inhibitory nucleic acid molecule that hybridizes to a KNTC1 nucleic acid molecule. Examples of inhibitory nucleic acid molecules include, but are not limited to, antisense nucleic acid molecules, small interfering RNAs (siRNAs), and short hairpin RNAs (shRNAs). Such inhibitory nucleic acid molecules can be designed to target any region of a KNTC1 nucleic acid molecule. In some embodiments, the antisense RNA, siRNA, or shRNA hybridizes to a sequence within a KNTC1 genomic nucleic acid molecule or mRNA molecule and decreases expression of the KNTC1 polypeptide in a cell in the subject. In some embodiments, the KNTC1 antagonist comprises an antisense molecule that hybridizes to a KNTC1 genomic nucleic acid molecule or mRNA molecule and decreases expression of the KNTC1 polypeptide in a cell in the subject. In some embodiments, the KNTC1 antagonist comprises an siRNA that hybridizes to a KNTC1 genomic nucleic acid molecule or mRNA molecule and decreases expression of the KNTC1 polypeptide in a cell in the subject. In some embodiments, the KNTC1 antagonist comprises an shRNA that hybridizes to a KNTC1 genomic nucleic acid molecule or mRNA molecule and decreases expression of the KNTC1 polypeptide in a cell in the subject.

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×His or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

In any of the embodiments described herein, any of the inhibitory nucleic acid molecules can be formulated as a component of a lipid nanoparticle, and can be delivered to a cell by a lipid nanoparticle.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (—OH, ═O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula: Sense: mN*mN*/i2FN/mN/i2FN/mN/i2FN/mN/i2FN/mN/i2FN/mN/i2FN/mN/i2FN/mN/i2FN/*mN*/32FN/Antisense: /52FN/*/i2FN/*mN/i2FN/mN/i2FN/mN/i2FN/mN/i2FN/mN/i2FN/mN/i2FN/mN/i2FN/mN/i2FN/mN*N*N

wherein: “N” is the base; “2F” is a 2′-F modification; “m” is a 2′-O-methyl modification, “I” is an internal base; and “*” is a phosphorothioate backbone linkage.

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the KNTC1 antagonist comprises a nuclease agent that induces one or more nicks or double-strand breaks at a recognition sequence(s) or a DNA-binding protein that binds to a recognition sequence within a KNTC1 genomic nucleic acid molecule. The recognition sequence can be located within a coding region of the KNTC1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the KNTC1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify the KNTC1 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of KNTC1 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in a KNTC1 genomic nucleic acid molecule or it can be a nickase that creates a single-strand break in a KNTC1 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of KNTC1 genomic nucleic acid molecules can be generated by contacting a cell with a Cas protein and one or more gRNAs that hybridize to one or more gRNA recognition sequences within a target genomic locus in the KNTC1 genomic nucleic acid molecule. For example, a KNTC1 gRNA recognition sequence can be located within a region of the KNTC1 genomic nucleic acid molecule. The gRNA recognition sequence can include or be proximate to the start codon of a KNTC1 genomic nucleic acid molecule or the stop codon of a KNTC1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in a KNTC1 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2-6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10, about 2 to about 5 base pairs, or three base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′-CCN-3′, where N is any DNA nucleotide and is immediately 5′ of the gRNA recognition sequence of the complementary strand of the target DNA.

A gRNA is an RNA molecule that binds to a Cas protein and targets the Cas protein to a specific location within a KNTC1 genomic nucleic acid molecule. An exemplary gRNA is a gRNA effective to direct a Cas enzyme to bind to or cleave a KNTC1 genomic nucleic acid molecule, wherein the gRNA comprises a DNA-targeting segment that hybridizes to a gRNA recognition sequence within the KNTC1 genomic nucleic acid molecule. Exemplary gRNAs comprise a DNA-targeting segment that hybridizes to a gRNA recognition sequence present within a KNTC1 genomic nucleic acid molecule that includes or is proximate to the start codon or the stop codon. For example, a gRNA can be selected such that it hybridizes to a gRNA recognition sequence that is located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the start codon or located from about 5, from about 10, from about 15, from about 20, from about 25, from about 30, from about 35, from about 40, from about 45, from about 50, from about 100, from about 200, from about 300, from about 400, from about 500, or from about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

The Cas protein and the gRNA form a complex, and the Cas protein cleaves the target KNTC1 genomic nucleic acid molecule. The Cas protein can cleave the nucleic acid molecule at a site within or outside of the nucleic acid sequence present in the target KNTC1 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the KNTC1 genomic nucleic acid molecule to which a DNA-targeting segment of a gRNA will bind.

Such methods can result, for example, in a KNTC1 genomic nucleic acid molecule in which a region of the gene is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the KNTC1 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the methods further comprising detecting the presence or absence of a KNTC1 variant nucleic acid molecule in a biological sample from the subject. In some embodiments, the KNTC1 variant nucleic acid molecule is a missense variant, splice-site variant, a stop-gain variant, a start-loss variant, a stop-loss variant, a frameshift variant, or an in-frame indel variant, a variant that encodes a loss-of-function polypeptide, a variant that encodes a truncated predicted loss-of-function polypeptide. In some embodiments, the KNTC1 variant nucleic acid molecule is a missense variant. In some embodiments, the KNTC1 variant nucleic acid molecule is a splice-site variant. In some embodiments, the KNTC1 variant nucleic acid molecule is a stop-gain variant. In some embodiments, the KNTC1 variant nucleic acid molecule is a start-loss variant. In some embodiments, the KNTC1 variant nucleic acid molecule is a stop-loss variant. In some embodiments, the KNTC1 variant nucleic acid molecule is a frameshift variant. In some embodiments, the KNTC1 variant nucleic acid molecule is an in-frame indel variant. In some embodiments, the KNTC1 variant nucleic acid molecule is a variant that encodes a loss-of-function polypeptide. In some embodiments, the KNTC1 variant nucleic acid molecule is a variant that encodes a truncated predicted loss-of-function polypeptide. In some embodiments, the KNTC1 variant nucleic acid molecule comprises the C>T rs61751321 single nucleotide polymorphism (chr12:122547931:C:T (GRCh38); chr12:123032478:C:T (GRCh37); see, for example, world wide web at “ncbi.nlm.nih.gov/snp/?term=rs61751321”).

In some embodiments, the methods further comprising detecting the presence or absence of an RC3H1 variant nucleic acid molecule in a biological sample from the subject. In some embodiments, the RC3H1 variant nucleic acid molecule is a missense variant, splice-site variant, a stop-gain variant, a start-loss variant, a stop-loss variant, a frameshift variant, or an in-frame indel variant, a variant that encodes a loss-of-function polypeptide, a variant that encodes a truncated predicted loss-of-function polypeptide. In some embodiments, the RC3H1 variant nucleic acid molecule is a missense variant. In some embodiments, the RC3H1 variant nucleic acid molecule is a splice-site variant. In some embodiments, the RC3H1 variant nucleic acid molecule is a stop-gain variant. In some embodiments, the RC3H1 variant nucleic acid molecule is a start-loss variant. In some embodiments, the RC3H1 variant nucleic acid molecule is a stop-loss variant. In some embodiments, the RC3H1 variant nucleic acid molecule is a frameshift variant. In some embodiments, the RC3H1 variant nucleic acid molecule is an in-frame indel variant. In some embodiments, the RC3H1 variant nucleic acid molecule is a variant that encodes a loss-of-function polypeptide. In some embodiments, the RC3H1 variant nucleic acid molecule is a variant that encodes a truncated predicted loss-of-function polypeptide. In some embodiments, the RC3H1 variant nucleic acid molecule comprises the G>C rs1660263700 single nucleotide polymorphism (chr1:173969582:G:C (GRCh38); chr1:173938720:G:C (GRCh37); see, for example, world wide web at “ncbi.nlm.nih.gov/snp/?term=rs1660263700”).

In some embodiments, the methods further comprising detecting the presence or absence of a YLPM1 variant nucleic acid molecule in a biological sample from the subject. In some embodiments, the YLPM1 variant nucleic acid molecule is a missense variant, splice-site variant, a stop-gain variant, a start-loss variant, a stop-loss variant, a frameshift variant, or an in-frame indel variant, a variant that encodes a loss-of-function polypeptide, a variant that encodes a truncated predicted loss-of-function polypeptide. In some embodiments, the YLPM1 variant nucleic acid molecule is a missense variant. In some embodiments, the YLPM1 variant nucleic acid molecule is a splice-site variant. In some embodiments, the YLPM1 variant nucleic acid molecule is a stop-gain variant. In some embodiments, the YLPM1 variant nucleic acid molecule is a start-loss variant. In some embodiments, the YLPM1 variant nucleic acid molecule is a stop-loss variant. In some embodiments, the YLPM1 variant nucleic acid molecule is a frameshift variant. In some embodiments, the YLPM1 variant nucleic acid molecule is an in-frame indel variant. In some embodiments, the YLPM1 variant nucleic acid molecule is a variant that encodes a loss-of-function polypeptide. In some embodiments, the YLPM1 variant nucleic acid molecule is a variant that encodes a truncated predicted loss-of-function polypeptide.

In some embodiments, the methods further comprising detecting the presence or absence of an HLA-DRB5 variant nucleic acid molecule in a biological sample from the subject. In some embodiments, the HLA-DRB5 variant nucleic acid molecule is a missense variant, splice-site variant, a stop-gain variant, a start-loss variant, a stop-loss variant, a frameshift variant, or an in-frame indel variant, a variant that encodes a loss-of-function polypeptide, a variant that encodes a truncated predicted loss-of-function polypeptide. In some embodiments, the HLA-DRB5 variant nucleic acid molecule is a missense variant. In some embodiments, the HLA-DRB5 variant nucleic acid molecule is a splice-site variant. In some embodiments, the HLA-DRB5 variant nucleic acid molecule is a stop-gain variant. In some embodiments, the HLA-DRB5 variant nucleic acid molecule is a start-loss variant. In some embodiments, the HLA-DRB5 variant nucleic acid molecule is a stop-loss variant. In some embodiments, the HLA-DRB5 variant nucleic acid molecule is a frameshift variant. In some embodiments, the HLA-DRB5 variant nucleic acid molecule is an in-frame indel variant. In some embodiments, the HLA-DRB5 variant nucleic acid molecule is a variant that encodes a loss-of-function polypeptide. In some embodiments, the HLA-DRB5 variant nucleic acid molecule is a variant that encodes a truncated predicted loss-of-function polypeptide. In some embodiments, the HLA-DRB5 variant nucleic acid molecule comprises the G>A rs774817822 single nucleotide polymorphism (chr6:32519439:G:A (GRCh38); chr6:32487216:G:A (GRCh37); see, for example, world wide web at “ncbi.nlm.nih.gov/snp/?term=rs774817822”).

The nucleotide sequence of a genomic wild-type KNTC1 gene (Ensembl version ENSG00000184445.12; chr12:122,527,246-122,626,396 forward strand, 99,151 nt (GRCh38.p13)) is set forth in, for example, the world wide web at “useast.ensembl.org/Homo_sapiens/Gene/Sequence?db=core;g=ENSG00000184445;r=12:122527246-122626396” with a 600 nt 5′ flanking sequence (upstream) added and a 600 nt 3′ flanking sequence (downstream) added. The rs61751321 variant (see, for example, world wide web at “ncbi.nlm.nih.gov/snp/rs61751321”) is a C:T substitution at chr12:122547931 (GRCh38.p13) and is a missense variant. Before this disclosure, the rs61751321 variant was of unknown clinical significance. In some embodiments, the rs61751321 variant can be a germline rs61751321 variant. In some embodiments, the rs61751321 variant can be a somatic rs61751321 variant.

The nucleotide sequence of a genomic wild-type RC3H1 gene (Ensembl version ENSG00000135870.12; chr1:173,931,084-174,022,357 reverse strand, 91,274 nt (GRCh38.p13)) is set forth in, for example, the world wide web at “useast.ensembl.org/Homo_sapiens/Gene/Sequence?db=core;g=ENSG00000135870;r=1:173931084-174022357” with a 600 nt 5′ flanking sequence (upstream) added and a 600 nt 3′ flanking sequence (downstream) added. The rs1660263700 variant (see, for example, world wide web at “www.ncbi.nlm.nih.gov/snp/rs1660263700”) is a G:C substitution at chr1:173969582 (GRCh38.p13) and is an intron variant. Before this disclosure, the rs1660263700 variant was of unknown clinical significance. In some embodiments, the rs1660263700 variant can be a germline RC3H1 variant. In some embodiments, the rs1660263700 variant can be a somatic RC3H1 variant.

The nucleotide sequence of a genomic wild-type YLPM1 gene (Ensembl version ENSG00000119596.18; chr14:74,763,316-74,859,435 forward strand, 96,120 nt (GRCh38.p13)) is set forth in, for example, the world wide web at “useast.ensembl.org/Homo_sapiens/Gene/Sequence?db=core;g=ENSG00000119596;r=14:74763316-74859435” with a 600 nt 5′ flanking sequence (upstream) added and a 600 nt 3′ flanking sequence (downstream) added.

The nucleotide sequence of a genomic wild-type HLA-DRB5 gene (Ensembl version ENSG00000198502.6; chr6:32,517,353-32,530,287 reverse strand, 12,935 nt (GRCh38.p13)) is set forth in, for example, the world wide web at “ensembl.org/Homo_sapiens/Gene/Sequence? g=ENSG00000198502;r=6:32517353-32530287;t=ENST00000374975” with a 600 nt 5′ flanking sequence (upstream) added and a 600 nt 3′ flanking sequence (downstream) added. The rs774817822 variant (see, for example, world wide web at “ncbi.nlm.nih.gov/snp/rs774817822”) is a G:A substitution at chr6:32519439 (GRCh38.p13) and is a stop-gain variant. Before this disclosure, the rs774817822 variant was of unknown clinical significance. In some embodiments, the rs774817822 variant can be a germline HLA-DRB5 variant. In some embodiments, the rs774817822 variant can be a somatic HLA-DRB5 variant.

The biological sample can be derived from any cell, tissue, or biological fluid from the subject. The biological sample may comprise any clinically relevant tissue, such as lung tissue or lung cells, such as from a biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample.

In some embodiments, the methods further comprise administering a therapeutic agent that treats, prevents, or reduces development of CH in a dosage amount that is the same or greater than a standard dosage amount to a subject wherein a KNTC1 variant nucleic acid molecule is absent from the biological sample. In some embodiments, the methods further comprise administering a therapeutic agent that treats, prevents, or reduces development of CH in a dosage amount that is the same or less than a standard dosage amount to a subject wherein a KNTC1 variant nucleic acid molecule is present in the biological sample. In some embodiments, the methods further comprise administering a therapeutic agent that treats, prevents, or reduces development of CH in a dosage amount that is the same as or less than a standard dosage amount to a subject that is heterozygous for a KNTC1 variant nucleic acid molecule. In some embodiments, the methods further comprise administering a therapeutic agent that treats, prevents, or reduces development of CH in a dosage amount that is the same as or less than a standard dosage amount to a subject that is homozygous for a KNTC1 variant nucleic acid molecule. In some embodiments, the subject comprises mLOY and/or mCA. In some embodiments, the subject comprises mLOY. In some embodiments, the subject comprises mCA.

In some embodiments, the methods further comprise administering a therapeutic agent that treats, prevents, or reduces development of CH in a dosage amount that is the same or less than a standard dosage amount to a subject wherein an RC3H1 variant nucleic acid molecule is absent from the biological sample. In some embodiments, the methods further comprise administering a therapeutic agent that treats, prevents, or reduces development of CH in a dosage amount that is the same or greater than a standard dosage amount to a subject wherein an RC3H1 variant nucleic acid molecule is present in the biological sample. In some embodiments, the methods further comprise administering a therapeutic agent that treats, prevents, or reduces development of CH in a dosage amount that is the same as or greater than a standard dosage amount to a subject that is heterozygous for an RC3H1 variant nucleic acid molecule. In some embodiments, the methods further comprise administering a therapeutic agent that treats, prevents, or reduces development of CH in a dosage amount that is the same as or greater than a standard dosage amount to a subject that is homozygous for an RC3H1 variant nucleic acid molecule. In some embodiments, the subject comprises mLOY and/or mCA. In some embodiments, the subject comprises mLOY. In some embodiments, the subject comprises mCA.

In some embodiments, the methods further comprise administering a therapeutic agent that treats, prevents, or reduces development of CH in a dosage amount that is the same or less than a standard dosage amount to a subject wherein a YLPM1 variant nucleic acid molecule is absent from the biological sample. In some embodiments, the methods further comprise administering a therapeutic agent that treats, prevents, or reduces development of CH in a dosage amount that is the same or greater than a standard dosage amount to a subject wherein a YLPM1 variant nucleic acid molecule is present in the biological sample. In some embodiments, the methods further comprise administering a therapeutic agent that treats, prevents, or reduces development of CH in a dosage amount that is the same as or greater than a standard dosage amount to a subject that is heterozygous for a YLPM1 variant nucleic acid molecule. In some embodiments, the methods further comprise administering a therapeutic agent that treats, prevents, or reduces development of CH in a dosage amount that is the same as or greater than a standard dosage amount to a subject that is homozygous for a YLPM1 variant nucleic acid molecule. In some embodiments, the subject comprises mLOY and/or mCA. In some embodiments, the subject comprises mLOY. In some embodiments, the subject comprises mCA.

In some embodiments, the methods further comprise administering a therapeutic agent that treats, prevents, or reduces development of LTL reduction in a dosage amount that is the same or less than a standard dosage amount to a subject wherein an HLA-DRB5 variant nucleic acid molecule is absent from the biological sample. In some embodiments, the methods further comprise administering a therapeutic agent that treats, prevents, or reduces development of LTL reduction in a dosage amount that is the same or greater than a standard dosage amount to a subject wherein an HLA-DRB5 variant nucleic acid molecule is present in the biological sample. In some embodiments, the methods further comprise administering a therapeutic agent that treats, prevents, or reduces development of LTL reduction in a dosage amount that is the same as or greater than a standard dosage amount to a subject that is heterozygous for an HLA-DRB5 variant nucleic acid molecule. In some embodiments, the methods further comprise administering a therapeutic agent that treats, prevents, or reduces development of LTL reduction in a dosage amount that is the same as or greater than a standard dosage amount to a subject that is homozygous for an HLA-DRB5 variant nucleic acid molecule.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in CH or LTL, a decrease/reduction in the severity of CH or LTL (such as, for example, a reduction or inhibition of development of CH or LTL), a decrease/reduction in symptoms and CH- or LTL-related effects, delaying the onset of symptoms and CH- or LTL-related effects, reducing the severity of symptoms of CH- or LTL-related effects, reducing the number of symptoms and CH- or LTL-related effects, reducing the latency of symptoms and CH- or LTL-related effects, an amelioration of symptoms and CH- or LTL-related effects, reducing secondary symptoms, reducing secondary infections, preventing relapse to CH or LTL, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. In some embodiments, the subject comprises mLOY and/or mCA. In some embodiments, the subject comprises mLOY. In some embodiments, the subject comprises mCA. In some embodiments, the CH-related effects comprise an mLOY-related effect and/or an mCA-related effect. In some embodiments, the CH-related effects comprise an mLOY-related effect. In some embodiments, the CH-related effects comprise an mCA-related effect. A prophylactic effect may comprise a complete or partial avoidance/inhibition or a delay of a CH or LTL reduction development/progression (such as, for example, a complete or partial avoidance/inhibition or a delay), and an increased survival time of the affected host animal, following administration of a therapeutic protocol. Treatment of CH or LTL encompasses the treatment of a subject already diagnosed as having any form of CH or LTL at any clinical stage or manifestation, the delay of the onset or evolution or aggravation or deterioration of the symptoms or signs of CH or LTL, and/or preventing and/or reducing the severity of CH or LTL.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45° C., followed by a wash of 2×SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The nucleic acid molecules can be from any organism. For example, the nucleic acid molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The present disclosure also provides methods of identifying a subject having an increased risk of developing CH. These methods comprise determining or having determined the presence or absence of a KNTC1 variant nucleic acid molecule in a biological sample obtained from the subject. When the subject is KNTC1 reference, then the subject has an increased risk of developing CH compared to a subject that comprises the KNTC1 variant nucleic acid molecule. When the subject is heterozygous or homozygous for the KNTC1 variant nucleic acid molecule, then the subject has a decreased risk of developing CH compared to a subject that is KNTC1 reference. In some embodiments, when the subject is KNTC1 reference, the subject is administered or continued to be administered a therapeutic agent that treats, prevents, or reduces development of CH in an amount that is the same as or greater than a standard dosage amount, and/or is administered a KNTC1 antagonist. In some embodiments, when the subject is heterozygous for a KNTC1 variant nucleic acid molecule, the subject is administered or continued to be administered a therapeutic agent that treats, prevents, or reduces development of CH in an amount that is the same as or less than a standard dosage amount, and/or is administering a KNTC1 antagonist. In some embodiments, when the subject is homozygous for a KNTC1 variant nucleic acid molecule, the subject is administered or continued to be administered a therapeutic agent that treats, prevents, or reduces development of CH in an amount that is the same as or less than a standard dosage amount, and/or is administered a KNTC1 antagonist. In some embodiments, the subject comprises mLOY and/or mCA. In some embodiments, the subject comprises mLOY. In some embodiments, the subject comprises mCA.

The present disclosure also provides methods of identifying a subject having an increased risk of developing CH. These methods comprise determining or having determined the presence or absence of an RC3H1 variant nucleic acid molecule in a biological sample obtained from the subject. When the subject is RC3H1 reference, then the subject has a decreased risk of developing CH compared to a subject that comprises the RC3H1 variant nucleic acid molecule. When the subject is heterozygous or homozygous for the RC3H1 variant nucleic acid molecule, then the subject has an increased risk of developing CH compared to a subject that is RC3H1 reference. In some embodiments, when the subject is RC3H1 reference, the subject is administered or continued to be administered a therapeutic agent that treats, prevents, or reduces development of CH in an amount that is the same as or less than a standard dosage amount, and/or is administered an RC3H1 agonist. In some embodiments, when the subject is heterozygous for an RC3H1 variant nucleic acid molecule, the subject is administered or continued to be administered a therapeutic agent that treats, prevents, or reduces development of CH in an amount that is the same as or greater than a standard dosage amount, and/or is administering an RC3H1 agonist. In some embodiments, when the subject is homozygous for an RC3H1 variant nucleic acid molecule, the subject is administered or continued to be administered a therapeutic agent that treats, prevents, or reduces development of CH in an amount that is the same as or greater than a standard dosage amount, and/or is administered an RC3H1 agonist. In some embodiments, the subject comprises mLOY and/or mCA. In some embodiments, the subject comprises mLOY. In some embodiments, the subject comprises mCA.

The present disclosure also provides methods of identifying a subject having an increased risk of developing CH. These methods comprise determining or having determined the presence or absence of a YLPM1 variant nucleic acid molecule in a biological sample obtained from the subject. When the subject is YLPM1 reference, then the subject has a decreased risk of developing CH compared to a subject that comprises the YLPM1 variant nucleic acid molecule. When the subject is heterozygous or homozygous for the YLPM1 variant nucleic acid molecule, then the subject has an increased risk of developing CH compared to a subject that is YLPM1 reference. In some embodiments, when the subject is YLPM1 reference, the subject is administered or continued to be administered a therapeutic agent that treats, prevents, or reduces development of CH in an amount that is the same as or less than a standard dosage amount, and/or is administered a YLPM1 agonist. In some embodiments, when the subject is heterozygous a YLPM1 variant nucleic acid molecule, the subject is administered or continued to be administered a therapeutic agent that treats, prevents, or reduces development of CH in an amount that is the same as or greater than a standard dosage amount, and/or is administering a YLPM1 agonist. In some embodiments, when the subject is homozygous for a YLPM1 variant nucleic acid molecule, the subject is administered or continued to be administered a therapeutic agent that treats, prevents, or reduces development of CH in an amount that is the same as or greater than a standard dosage amount, and/or is administered a YLPM1 agonist. In some embodiments, the subject comprises mLOY and/or mCA. In some embodiments, the subject comprises mLOY. In some embodiments, the subject comprises mCA.

The present disclosure also provides methods of identifying a subject having an increased risk of developing LTL reduction. These methods comprise determining or having determined the presence or absence of an HLA-DRB5 variant nucleic acid molecule in a biological sample obtained from the subject. When the subject is HLA-DRB5 reference, then the subject has a decreased risk of developing LTL reduction compared to a subject that comprises the HLA-DRB5 variant nucleic acid molecule. When the subject is heterozygous or homozygous for the HLA-DRB5 variant nucleic acid molecule, then the subject has an increased risk of developing LTL reduction compared to a subject that is HLA-DRB5 reference. In some embodiments, when the subject is HLA-DRB5 reference, the subject is administered or continued to be administered a therapeutic agent that treats, prevents, or reduces development of LTL reduction in an amount that is the same as or less than a standard dosage amount, and/or is administered an HLA-DRB5 agonist. In some embodiments, when the subject is heterozygous an HLA-DRB5 variant nucleic acid molecule, the subject is administered or continued to be administered a therapeutic agent that treats, prevents, or reduces development of LTL reduction in an amount that is the same as or greater than a standard dosage amount, and/or is administering an HLA-DRB5 agonist. In some embodiments, when the subject is homozygous for an HLA-DRB5 variant nucleic acid molecule, the subject is administered or continued to be administered a therapeutic agent that treats, prevents, or reduces development of LTL reduction in an amount that is the same as or greater than a standard dosage amount, and/or is administered an HLA-DRB5 agonist.

In any of the embodiments described herein, the KNTC1 variant nucleic acid molecule can be any KNTC1 variant nucleic acid molecule described herein. In any of the embodiments described herein, the RC3H1 variant nucleic acid molecule can be any RC3H1 variant nucleic acid molecule described herein. In any of the embodiments described herein, the YLPM1 variant nucleic acid molecule can be any YLPM1 variant nucleic acid molecule described herein. In any of the embodiments described herein, the HLA-DRB5 variant nucleic acid molecule can be any HLA-DRB5 variant nucleic acid molecule described herein.

In any of the embodiments described herein, the KNTC1 antagonist can be any of the KNTC1 antagonists described herein. In any of the embodiments described herein, the RC3H1 agonist can be any of the RC3H1 agonists described herein. In any of the embodiments described herein, the YLPM1 agonist can be any of the YLPM1 agonists described herein. In any of the embodiments described herein, the HLA-DRB5 agonist can be any of the HLA-DRB5 agonists described herein.

The present disclosure also provides uses of any of the therapeutic agents that prevent or reduce CH described herein for use in preventing or treating (or for use in the preparation of a medicament for preventing or treating) CH in a subject. In some embodiments, the subject comprises mLOY and/or mCA. In some embodiments, the subject comprises mLOY. In some embodiments, the subject comprises mCA.

The present disclosure also provides uses of any of the therapeutic agents that prevent or reduce LTL reduction described herein for use in preventing or treating (or for use in the preparation of a medicament for preventing or treating) LTL reduction in a subject.

The present disclosure also provides uses of any of the KNTC1 antagonists, RC3H1 agonists, and/or YLPM1 agonists described herein for use in preventing or treating (or for use in the preparation of a medicament for preventing or treating) CH in a subject. In some embodiments, the subject comprises mLOY and/or mCA. In some embodiments, the subject comprises mLOY. In some embodiments, the subject comprises mCA.

The present disclosure also provides uses of any of the HLA-DRB5 agonists described herein for use in preventing or treating (or for use in the preparation of a medicament for preventing or treating) LTL reduction in a subject.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in ° C. or is at ambient temperature, and pressure is at or near atmospheric.

### EXAMPLES

### Methods

Exome sequencing data were used to characterize CHIP status in 454,803 UK Biobank (UKB) participants (Backman et al., Nature, 2021, 599, 628-34) and 173,585 Geisinger MyCode Community Health Initiative (GHS) participants. A common variant genome-wide association study (GWAS) and rare variant and gene burden exome-wide association study (ExWAS) of CHIP in 27,331 CHIP mutation carriers from the UKB was then conducted. A replication analysis in 12,877 CHIP mutation carriers from the GHS cohort was performed. To identify germline predictors of specific clonal hematopoiesis (CH) driver mutations, GWAS and ExWAS in carriers of CHIP mutations in individual CHIP genes was also conducted. Genetic association findings for CHIP were then compared to those from analyses of other CH phenotypes determined from somatic alterations in the blood, including mCA, mosaic loss of sex chromosomes (mLOX and mLOY) and telomere length were then compared. While GWAS of these non-CHIP clonal hematopoiesis phenotypes have been conducted before (Zekavat et al., Nat. Medicine, 2021, 27, 1012-24; Thompson et al., Nature, 2019, 575, 652-57; Codd et al., Nat. Genet., 2021, 53, 1425-33), none have evaluated the impact of very rare variants (minor allele frequency (MAF) 0.005). The ExWAS performed represents the first systematic large-scale exploration of rare variants on the genetic susceptibility of these phenotypes.

ExWAS Analysis of Mosaic Chromosomal Alteration (mCA) Phenotypes

Rare variant and gene burden associations analyses for the mLOY, mLOX, and autosomal mCA phenotypes were performed, which exclude samples with CHIP mutations and should therefore be mCA specific. Notably, a novel risk reducing association between a rare missense variant in the KNTC1 gene (rs61751321-T, alternative allele frequency (AAF)=0.003, L317F) and the mLOY phenotype (OR=0.60 (0.50-0.72), P=2.56×10⁻⁸) was found. While this association was below the strict Bonferroni multiple-testing correction threshold used for rare variants (P<=7.14×10⁻¹⁰), the association was interesting. Given that the KNTC1 gene shows lymphocyte specific expression (data not shown), and that this rare missense variant was predicted to be deleterious by >2 computational predictors (e.g., Combined Annotation-Dependent Depletion (CADD) and Sorting Intolerant from Tolerant (SIFT)), it is plausible that this association represents a genetic loss of function that antagonizes the clonal hematopoietic expansion that accompanies mLOY. Using a gene burden framework (i.e., ‘M1’ burden masks; Codd et al., Nat. Genet., 2021, 53, 1425-33), a rare variant signal was also identified supporting a recently described (Denny et al., Bioinformatics, 2010, 26, 1205-10) risk increasing association between rare loss of function variants in the GIGYF1 gene and mLOY (5.61 (3.35-9.40), P=5.73×10⁻¹¹). The data obtained for the KNTC1 gene were: 12:122547931:C:T (name), 12 (chr), 122547931 (pos), C (ref), T (alt), mLOY_exclusive (trait), ADD-WGR-FIRTH (model), 0.602034 (effect), 0.503571 (Ici_effect), 0.71975 (uci_effect), 2.56E-08 (pval), 0.00299479 (aaf), 35406 (num_cases), 35261 (cases_ref), 145 (cases_het), 0 (cases_alt), 141735 (num_controls), 140821 (controls_ref), 912 (controls_het), 2 (controls_alt), missense (variantEffect), FALSE (isLof), and KNTC1 (nearestGene).

A novel association was found between rare loss-of-function variation in the RC3H1 gene (OR=44 (16-127), P=1.16×10⁻¹²) and mCAs on chromosome 1, suggesting RC3H1 as a gene that drives CH. Variant allele fraction (VAF) and age associations suggested these variants were germline, and that are similar to the MPL gene, the RC3H1 gene is biallelically lost when mCAs disrupt the remaining functional gene copy. The data obtained for the RC3H1 gene were RC3H1 (gene), M1.0001 (mask), 1 (chr), 173938720 (pos), mCAaut_chr1_exclusive (trait), 44.77 (15.70,127.65) (or (95% ci)), 1,096 1910 (genotype counts (cases)), 342,81615310 (genotype counts (controls)), 1.16E-12 (pval), 9.00E-05 (aaf), 0.233 (VAF), 1.005 (Beta_Age), and 0.7266 (Pval_Age).

Rare loss-of-function variants in the YLPM1 gene that were strongly age-associated and likely somatic in origin were associated with mCAs on chromosome 14 (OR=30 (12-75), P=2.44×10⁻¹³). The data obtained for the YLPM1 gene were YLPM1 (gene), M1.0001 (mask), 14 (chr), 74763489 (pos), mCAaut_chr14_exclusive (trait), 30.18 (12.12,75.12) (or (95% ci)), 70411110 (genotype counts (cases)), 342,7221 14710 (genotype counts (controls)), 2.44E-13 (pval), 0.00023 (aaf), 0.698 (VAF), 1.033 (Beta_Age), and 0.000317 (Pval_Age). The positions are with respect to GRCh38.

### ExWAS Analysis of Telomere Length

An ExWAS of leukocyte telomere length, as quantified by Codd et al., Nat. Genet., 2021, 53, 1425-33, conditioned on common variant signals identified by GWAS was also performed. A rare and highly likely to be damaging loss-of-function variant in the HLA-DRB5 gene (rs774817822-A, MAF=9.56×10⁻⁶, MAC=8, CADD=35) was identified that was associated with nearly a three standardized unit reduction in telomere length (−2.23 (−2.89-−1.57), P=3.23×10⁻¹¹). A phenome-wide association studies (PheWAS) of these two rare variants did not identify any significant associations, although the PheWAS was notably underpowered due to the low MAC of these variants. The data obtained for the HLA-DRB5 gene were −3.57362 (Ici_effect), −1.90866 (uci_effect), 1.09E-10 (pval), 6.02E-06 (aaf), 415615 (num_cases), 415610 (cases_ref), 5 (cases_het), 0 (cases_alt), NA (num_controls), NA (controls_ref), NA (controls_het), NA (controls_alt), stop_gained (varianteffect), TRUE (islof), HLA-DRB5 (nearestgene), 0.2 (VAF), 1.096 (Beta_Age), and 0.1945 (Pval_Age).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating, preventing, or reducing the development of clonal hematopoiesis (CH) in a subject, the method comprising administering at least one kinetochore associated 1 (KNTC1) antagonist to the subject.

2-3. (canceled)

4. The method according to claim 1, wherein the KNTC1 antagonist comprises an antisense nucleic acid molecule, a small interfering RNA (siRNA), or a short hairpin RNA (shRNA) that hybridizes to a KNTC1 nucleic acid molecule.

5-15. (canceled)

16. A method of treating, preventing, or reducing the development of clonal hematopoiesis (CH) in a subject, the method comprising administering at least one ring finger and CCCH-type domains 1 (RC3H1) agonist to the subject.

17-18. (canceled)

19. The method according to claim 16, wherein the RC3H1 agonist comprises an RC3H1 protein.

20-25. (canceled)

26. A method of treating, preventing, or reducing the development of clonal hematopoiesis (CH) in a subject, the method comprising administering at least one YLP motif containing 1 (YLPM1) agonist to the subject.

27-28. (canceled)

29. The method according to claim 26, wherein the YLPM1 agonist comprises a YLPM1 protein.

30-35. (canceled)

36. A method of treating, preventing, or reducing the development of leukocyte telomere length (LTL) reduction in a subject, the method comprising administering at least one Major Histocompatibility Complex, Class II, DR beta 5 (HLA-DRB5) agonist.

37. (canceled)

38. The method according to claim 36, wherein the HLA-DRB5 agonist comprises an HLA-DRB5 protein.

39-44. (canceled)

45. A method of treating a subject with a therapeutic agent that treats, prevents, or reduces development of clonal hematopoiesis (CH), wherein the subject has CH or is at risk of developing CH, the method comprising:
determining whether the subject has a kinetochore associated 1 (KNTC1) variant nucleic acid molecule by:
obtaining or having obtained a biological sample from the subject; and
performing or having performed a sequence analysis on the biological sample to determine if the subject has a genotype comprising a KNTC1 variant nucleic acid molecule; and
administering or continuing to administer the therapeutic agent that treats, prevents, or reduces development of CH in an amount that is the same as or greater than a standard dosage amount to a subject that is KNTC1 reference; and/or administering a KNTC1 antagonist to the subject;
administering or continuing to administer the therapeutic agent that treats, prevents, or reduces development of CH in an amount that is the same as or less than a standard dosage amount to a subject that is heterozygous for the KNTC1 variant nucleic acid molecule; and/or administering a KNTC1 antagonist to the subject; or
administering or continuing to administer the therapeutic agent that treats, prevents, or reduces development of CH in an amount that is the same as or less than a standard dosage amount to a subject that is homozygous for the KNTC1 variant nucleic acid molecule; and/or administering a KNTC1 antagonist to the subject;
wherein the presence of a genotype having the KNTC1 variant nucleic acid molecule, indicates the subject has a decreased risk of developing CH.

46-49. (canceled)

50. The method according to claim 45, wherein the KNTC1 variant nucleic acid molecule is a missense variant, splice-site variant, a stop-gain variant, a start-loss variant, a stop-loss variant, a frameshift variant, an in-frame indel variant, a variant that encodes a loss-of-function polypeptide, or a variant that encodes a truncated predicted loss-of-function polypeptide.

51. (canceled)

52. The method according to claim 45, wherein the KNTC1 antagonist comprises an antisense nucleic acid molecule, a small interfering RNA (siRNA), or a short hairpin RNA (shRNA) that hybridizes to a KNTC1 nucleic acid molecule.

53-57. (canceled)

58. A method of treating a subject with a therapeutic agent that treats, prevents, or reduces development of clonal hematopoiesis (CH), wherein the subject has CH or is at risk of developing CH, the method comprising:
determining whether the subject has a ring finger and CCCH-type domains 1 (RC3H1) variant nucleic acid molecule by:
obtaining or having obtained a biological sample from the subject; and
performing or having performed a sequence analysis on the biological sample to determine if the subject has a genotype comprising an RC3H1 variant nucleic acid molecule; and
administering or continuing to administer the therapeutic agent that treats, prevents, or reduces development of CH in an amount that is the same as or less than a standard dosage amount to a subject that is RC3H1 reference; and/or administering an RC3H1 agonist to the subject;
administering or continuing to administer the therapeutic agent that treats, prevents, or reduces development of CH in an amount that is the same as or greater than a standard dosage amount to a subject that is heterozygous for the RC3H1 variant nucleic acid molecule; and/or administering an RC3H1 agonist to the subject; or
administering or continuing to administer the therapeutic agent that treats, prevents, or reduces development of CH in an amount that is the same as or greater than a standard dosage amount to a subject that is homozygous for the RC3H1 variant nucleic acid molecule; and/or administering an RC3H1 agonist to the subject;
wherein the presence of a genotype having the RC3H1 variant nucleic acid molecule indicates the subject has an increased risk of developing CH.

59-62. (canceled)

63. The method according to claim 58, wherein the RC3H1 variant nucleic acid molecule is a missense variant, splice-site variant, a stop-gain variant, a start-loss variant, a stop-loss variant, a frameshift variant, an in-frame indel variant, a variant that encodes a loss-of-function polypeptide, or a variant that encodes a truncated predicted loss-of-function polypeptide.

64. (canceled)

65. The method according to claim 58, wherein the RC3H1 agonist comprises an RC3H1 protein.

66. A method of treating a subject with a therapeutic agent that treats, prevents, or reduces development of clonal hematopoiesis (CH), wherein the subject has CH or is at risk of developing CH, the method comprising:
determining whether the subject has a YLP motif containing 1 (YLPM1) variant nucleic acid molecule by:
obtaining or having obtained a biological sample from the subject; and
performing or having performed a sequence analysis on the biological sample to determine if the subject has a genotype comprising a YLPM1 variant nucleic acid molecule; and
administering or continuing to administer the therapeutic agent that treats, prevents, or reduces development of CH in an amount that is the same as or less than a standard dosage amount to a subject that is YLPM1 reference; and/or administering a YLPM1 agonist to the subject;
administering or continuing to administer the therapeutic agent that treats, prevents, or reduces development of CH in an amount that is the same as or greater than a standard dosage amount to a subject that is heterozygous for the YLPM1 variant nucleic acid molecule; and/or administering a YLPM1 agonist to the subject; or
administering or continuing to administer the therapeutic agent that treats, prevents, or reduces development of CH in an amount that is the same as or greater than a standard dosage amount to a subject that is homozygous for the YLPM1 variant nucleic acid molecule; and/or administering a YLPM1 agonist to the subject;
wherein the presence of a genotype having the YLPM1 variant nucleic acid molecule indicates the subject has an increased risk of developing CH.

67-70. (canceled)

71. The method according to claim 66, wherein the YLPM1 variant nucleic acid molecule is a missense variant, splice-site variant, a stop-gain variant, a start-loss variant, a stop-loss variant, a frameshift variant, an in-frame indel variant, a variant that encodes a loss-of-function polypeptide, or a variant that encodes a truncated predicted loss-of-function polypeptide.

72. (canceled)

73. The method according to claim 66, wherein the YLPM1 agonist comprises a YLPM1 protein.

74. A method of treating a subject with a therapeutic agent that treats, prevents, or reduces development of leukocyte telomere length (LTL) reduction, wherein the subject has LTL reduction or is at risk of developing LTL reduction, the method comprising:
determining whether the subject has a Major Histocompatibility Complex, Class II, DR beta 5 (HLA-DRB5) variant nucleic acid molecule by:
obtaining or having obtained a biological sample from the subject; and
performing or having performed a sequence analysis on the biological sample to determine if the subject has a genotype comprising an HLA-DRB5 variant nucleic acid molecule; and
administering or continuing to administer the therapeutic agent that treats, prevents, or reduces development of LTL reduction in an amount that is the same as or less than a standard dosage amount to a subject that is HLA-DRB5 reference; and/or administering an HLA-DRB5 agonist to the subject;
administering or continuing to administer the therapeutic agent that treats, prevents, or reduces development of LTL reduction in an amount that is the same as or greater than a standard dosage amount to a subject that is heterozygous for the HLA-DRB5 variant nucleic acid molecule; and/or administering an HLA-DRB5 agonist to the subject; or
administering or continuing to administer the therapeutic agent that treats, prevents, or reduces development of LTL reduction in an amount that is the same as or greater than a standard dosage amount to a subject that is homozygous for the HLA-DRB5 variant nucleic acid molecule; and/or administering an HLA-DRB5 agonist to the subject;
wherein the presence of a genotype having the HLA-DRB5 variant nucleic acid molecule indicates the subject has an increased risk of developing LTL.

75-77. (canceled)

78. The method according to claim 74, wherein the HLA-DRB5 variant nucleic acid molecule is a missense variant, splice-site variant, a stop-gain variant, a start-loss variant, a stop-loss variant, a frameshift variant, an in-frame indel variant, a variant that encodes a loss-of-function polypeptide, or a variant that encodes a truncated predicted loss-of-function polypeptide.

79. (canceled)

80. The method according to claim 74, wherein the HLA-DRB5 agonist comprises an HLA-DRB5 protein.

81-120. (canceled)
